(19) United States
(12) Patent Application Publication
(10) Pub. No.: US 2009/0182248 A1
(43) Pub. Date: Jul. 16, 2009

(54) Systems and methods for monitoring an activity of a patient

(75) Inventors: Vernon Thomas Jensen, Draper, UT (US); Christopher Allen Nafis, Rexford, NY (US)

Correspondence Address: Armstrong Teasdale LLP (12553 - 1000);Patrick W. Rasche, ONE METROPOLITAN SQUARE, SUITE 2600, ST. LOUIS, MO, 63102-2740, US

(73) Assignee: General Electric Company

(21) Appl. No.: 12/008,857

(22) Filed: Jan. 15, 2008

(51) Int. Cl. A61B 5/11

(52) U.S. Cl. 600/595

(57) ABSTRACT

A system for monitoring an activity of a patient is provided. The system includes at least one electromagnetic transmitter configured to transmit at least one electromagnetic signal, and at least one electromagnetic receiver configured to receive at least one electromagnetic signal. The system further includes a processor configured to determine at least one position and at least one orientation of at least one electromagnetic transmitter with respect to at least one electromagnetic receiver to determine the activity of the patient.

### BACKGROUND OF THE INVENTION

This invention relates generally to patient monitoring systems and methods and more particularly to systems and methods for monitoring an activity of a patient.

Sleep apnea, or sleep-disordered breathing, is a condition in which breathing of an individual is briefly interrupted or even stops episodically during sleep. Because repeated arousal or alternatively full awakening when breathing is interrupted disturbs sleep, the individual suffering from sleep apnea is often drowsy during the day. Additionally, the individual may suffer from a plurality of symptoms of sleep deprivation. Such symptoms may include daytime drowsiness, tiredness or fatigue, difficulties with mental concentration, mood changes, reductions in performance, increase in mistakes, and increased risk of accidents. Further, complications from an insufficient amount of oxygen reaching the brain of the individual are serious and potentially life threatening.

Sleep apnea appears to be far more common than was initially realized when it was first described in 1965. Approximately 6-7% of the population of the United States, or 18 million Americans, are thought to have sleep apnea, but approximately 10 million have symptoms. In Americans aged approximately 30-60 years, obstructive sleep apnea affects nearly one in four men and one in 10 women. Men are twice as likely to have sleep apnea.

An estimated 2,000,000 children or approximately 3% of the child population of the United States are also affected. In children, sleep apnea can be associated with excessive daytime sleepiness, hyperactivity, attention deficit disorder, poor hearing, physical debilitation, and failure to thrive.

When sleep apnea is suspected, an overnight polysomnography (PSG) testing at a specialized sleep center may be suggested to determine an existence of sleep apnea within the individual. During this test, breathing, brain waves, heartbeat, muscle tension, and eye movement of the individual are monitored through wires attached to the skin while the individual sleeps. After the test, a physician trained in PSG testing analyzes the monitored data to determine if sleep apnea or other conditions are present in the individual. However, PSG testing is costly.

In some cases, portable PSG testing can also be done at home after a sleep technologist attaches the wires and instructs a responsible adult on how to record sleep activity of the individual. Although portable PSG tests may be less expensive and more convenient than the overnight PSG testing mentioned above, the portable PSG tests are subject to lost or inadequate recording, technical problems, and a low diagnostic accuracy compared to the overnight PSG testing. Patients with inconclusive results on home studies and those with negative studies but persistent symptoms should have the overnight PSG testing in the sleep center.

Although imaging has been used in some studies to determine the root cause of an airway blockage within the individual, it is not practical for a larger population of patients, due to cost and availability issues. Moreover, oxygen levels of the individual can be monitored through a fingertip device applied to a fingertip of the individual to determine sleep apnea. However, the fingertip device creates additional sleep discomfort to the individual.

The individual may be placed under observation by using a video camera to determine whether the individual has sleep apnea. A medical personnel reviews video images generated by the video camera to determine whether the individual has sleep apnea.

However, the individual does not feel comfortable in being observed via the video images. The individual may feel that his/her privacy is invaded when observed via the video images. Moreover, the medical personnel reviews the video images generated by the video camera by operating a video tape, which takes a significant amount of time. For example, it takes time for the medical personnel to rewind or forward to a particular portion of the video tape.

To increase the effectiveness of determining movement of the individual, a plurality of optical reflectors could be attached to the individual and the video camera generates the video images of the individual with the optical reflectors. However, portions of the individual to which the optical reflectors are attached are not visible when the individual covers the reflectors with a cloth or a line-of-sight between the video camera and the optical reflectors is blocked.

Additionally, in some cases, a plurality of motion sensors are used to sense a motion of the individual. The medical personnel, without observing the individual, cannot identify, from a representation of a signal sensed by the motion sensors, a particular part of the individual that moves.

### BRIEF DESCRIPTION OF THE INVENTION

In one aspect, a system for monitoring an activity of a patient is provided. The system includes at least one electromagnetic transmitter configured to transmit at least one electromagnetic signal and at least one electromagnetic receiver configured to receive at least one electromagnetic signal. The system further includes a processor configured to determine at least one position and at least one orientation of at least one electromagnetic transmitter with respect to at least one electromagnetic receiver to determine the activity of the patient.

In another aspect, a system for monitoring an activity of a patient is described. The system includes at least one electromagnetic transmitter configured to transmit at least one electromagnetic signal and at least one electromagnetic receiver configured to receive at least one electromagnetic signal. The system further includes a processor configured to determine at least one mutual inductance between at least one electromagnetic transmitter and at least one electromagnetic receiver. The processor is configured to determine at least one position and at least one orientation of at least one electromagnetic transmitter with respect to at least one electromagnetic receiver based on at least one mutual inductance to determine the activity of the patient.

In yet another aspect, a method for monitoring an activity of a patient is described. The method includes transmitting at least one electromagnetic signal from at least one electromagnetic transmitter, receiving, by at least one electromagnetic receiver, at least one electromagnetic signal, and determining the activity of the patient by determining at least one position and at least one orientation of at least one electromagnetic transmitter with respect to at least one electromagnetic receiver.

### BRIEF DESCRIPTION OF THE DRAWINGS

FIG. 1 is an isometric view of an embodiment of a system for monitoring an activity of a patient.

FIG. 2 is an exemplary block diagram of an embodiment of a system for monitoring an activity of patient.

FIG. 3 is an isometric view of another embodiment of a system for monitoring an activity of patient.

FIG. 4 is an isometric view of yet another embodiment of a system for monitoring an activity of patient.

FIG. 5 is an isometric view of still another embodiment of a system for monitoring an activity of patient.

### DETAILED DESCRIPTION OF THE INVENTION

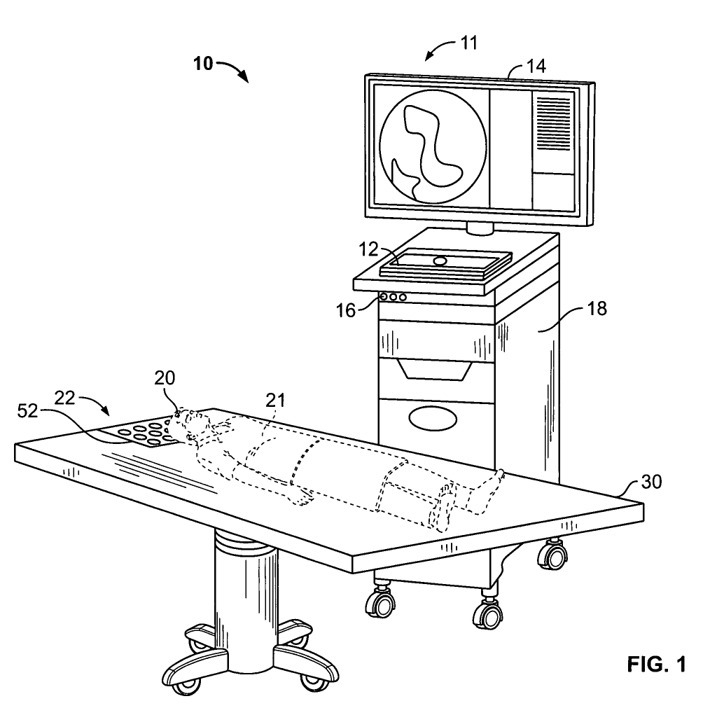

FIG. 1 is an isometric view of an embodiment of a system 10 for monitoring an activity of a patient. System 10 includes a medical navigation system 11 that further includes a portable computer 12, at least one display 14, and a navigation interface 16 on a portable cart 18. System 10 also includes at least one electromagnetic field generator 20 attached to a patient 21 and at least one electromagnetic field sensor 22.

At least one electromagnetic field generator 20 may be an active device or a passive device. Similarly, at least one electromagnetic field sensor 22 may be an active or a passive device. Moreover, at least one electromagnetic field generator 20 may be a fixed frequency, a multifrequency, or an adaptive frequency device. Similarly, at least one electromagnetic sensor 22 may be a fixed frequency, a multifrequency, or an adaptive frequency device.

At least one electromagnetic field generator 20 may be attached to patient 21 or a garment of patient 21 in a rigid or a flexible manner. Moreover, at least one electromagnetic field generator 20 may be coupled to navigation interface 16 via a wired or a wireless connection.

At least one electromagnetic sensor 22 may be configured on a printed circuit board. Certain embodiments may include at least one electromagnetic field sensor 22 including a printed circuit board receiver array 52 including a plurality of coil and coil pairs and electronics for digitizing magnetic measurements in the printed circuit board receiver array 52. At least one electromagnetic field sensor 22 may be attached to table 30, medical navigation system 11, a therapy system, a secondary monitoring device, or to portions, such as floor and walls, of a room in which patient 21 is situated, in a flexible or a rigid manner, such as via a rigid or flexible printed circuit board. The printed circuit board receiver array 52 is configurable. A user, such as a medical personnel, or an operator may swap out and use different configurations of printed circuit board receiver array 52 for different applications. A table 30 is positioned near at least one electromagnetic field sensor 22 to support patient 21.

At least one electromagnetic field generator 20 generates an electromagnetic field and at least one electromagnetic field sensor 22 detects the electromagnetic field to generate a set of electromagnetic field measurements. The medical navigation system 11 operates with at least one electromagnetic field generator 20 and at least one electromagnetic field sensor 22 to determine position and orientation information, such as at least one location of at least one electromagnetic field generator 20 with respect to at least one electromagnetic field sensor 22 and at least one orientation of at least one electromagnetic field generator 20 with respect to at least one electromagnetic field sensor 22. The electromagnetic field detected by at least one electromagnetic field sensor 22 is used to monitor not only dynamic body motion of patient 21 but several other events that affect sleep quality of patient 21.

The electromagnetic field measurements can be used to calculate the position and orientation information according to any suitable method or system. After the electromagnetic field measurements are digitized using electronics of at least one electromagnetic field sensor 22, the digitized signals are transmitted to the navigation interface 16 through a wireless connection. Alternatively, at least one electromagnetic sensor 22 may be coupled to the navigation interface 16 through a wired connection and the digitized signals are transmitted from at least one electromagnetic field sensor 22 to the navigation interface 16 via the wired connection. The medical navigation system 11 is used to calculate the position and orientation information of at least one electromagnetic transmitter 20 with respect to at least one electromagnetic sensor 22 based on at least one mutual inductance between at least one electromagnetic transmitter 20 and at least one electromagnetic sensor 22.

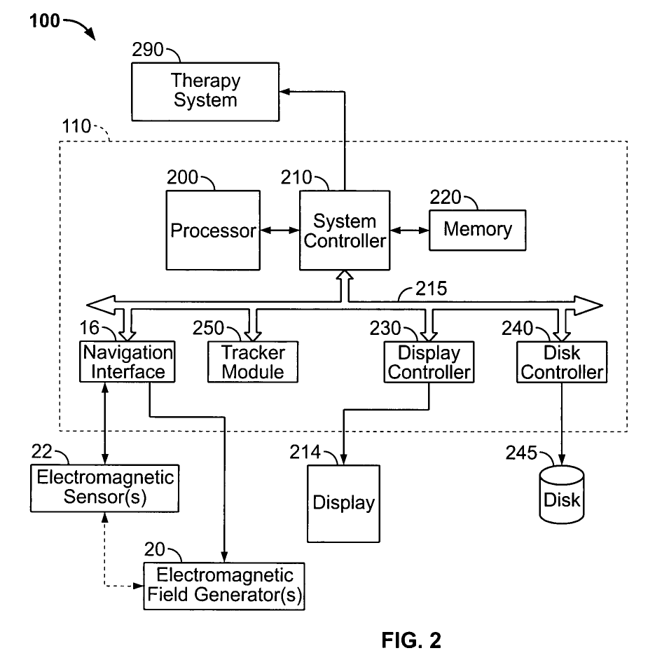

FIG. 2 is an exemplary block diagram of an embodiment of a system 100 for monitoring an activity of patient 21. System 100 includes a medical navigation system 110. The medical navigation system 110 is illustrated conceptually as a collection of modules, but may be implemented using any combination of dedicated hardware boards, digital signal processors, field programmable gate arrays, and processors. Alternatively, the modules may be implemented using an off-the-shelf computer with a single processor or multiple processors, with the functional operations distributed between the processors. As an example, it may be desirable to have a dedicated processor for the position and orientation information calculations as well as a dedicated processor for display operations. As a further option, the modules may be implemented using a hybrid configuration in which certain modular functions are performed using dedicated hardware, while the remaining modular functions are performed using an off-the-shelf computer. In the embodiment, shown in FIG. 2, the medical navigation system 110 includes a processor 200, a system controller 210, and a memory 220. Memory 220 may be an optical memory, a flash memory, or a magnetic memory. The operations of the modules may be controlled by system controller 210.

At least one electromagnetic field generator 20 is coupled to navigation interface 16. The system 100 may be configured to assign a unique identifier to each electromagnetic field generator 20 through the navigation interface 16, so that the system 100 can identify which electromagnetic field generator 20 is attached to which portion, such as leg or arms or head, of patient 21, or portion of a garment worn by patient 21. At least one electromagnetic field generator 20 generates at least one electromagnetic field that is detected by at least one electromagnetic sensor 22. In another embodiment, system 100 assigns a unique identifier to each electromagnetic sensor 22 that is attached to patient 21. The assigning of the unique identifier to each electromagnetic sensor 22 helps identify a particular electromagnetic sensor 22 attached to a particular portion, such as an arm, leg, or waist, of patient 21, or a portion of the garment worn by patient 21.

The navigation interface 16 receives digitized signals from at least one electromagnetic field sensor 22. In the embodiment illustrated in FIG. 1, the navigation interface 16 includes at least one Ethernet port. At least one Ethernet port may be provided, for example, with an Ethernet network interface card or adapter. However, according to various alternate embodiment, the digitized signals may be transmitted from at least one electromagnetic sensor 22 to the navigation interface 16 using alternative wired or wireless communication protocols and interfaces.

The digitized signals received by the navigation interface 16 represent magnetic field information from at least one electromagnetic field generator 20 detected by at least one electromagnetic field sensor 22. In the embodiment illustrated in FIG. 2, the navigation interface 16 transmits the digitized signals to a tracker module 250 over a local interface 215, such as a peripheral component interconnect (PCI) bus. According to various alternate embodiments, various equivalent bus technologies may be substituted.

The tracker module 250 calculates the position and orientation information based on the received digitized signals that represent at least one mutual inductance between at least one electromagnetic field generator 20 and at least one electromagnetic field sensor 22. For example, the tracker module 250 calculates a mutual inductance between an electromagnetic field generator 20 and an electromagnetic field sensor 22 from the digitized signals, and determines a position and an orientation of the electromagnetic field generator 20 with respect to the electromagnetic field sensor 22 from a look-up table stored within a memory 220 or a disk 245. The look-up table includes relationships between mutual inductances and positions and orientations.

The position and orientation information is stored by a system controller 210 in memory 220 and/or by a disk controller 240 into disk 245. As used herein, the term controller is not limited to just those integrated circuits referred to in the art as a controller, but broadly refers to a processor, a computer, a microcontroller, a microcomputer, a programmable logic controller, an application specific integrated circuit, and any other programmable circuit. The disk 245 and the memory 220 are examples of a computer-readable medium. By way of example only, the disk 245 is a hard disk but other suitable storage devices may be used. The disk controller 240 retrieves data from and stores data on disk 245.

The position and orientation information provides at least one location and at least one orientation of at least one electromagnetic field generator 20 with respect to at least one electromagnetic field sensor 22. The position and orientation information is used to create an in-depth understanding of dynamic body motion of patient 21.

System controller 210 and tracker module 250 translate the position and orientation information into a virtual stickman representing an activity or movement of patient 22, or an atlas based representation of body poses of patient 21. For example, if at least one electromagnetic field generator 20 is attached to limbs, such as arms and legs, of patient 21, the tracker module 250 generates the position and orientation information representing movement of the limbs of patient 21 from the electromagnetic signals generated by at least one electromagnetic field generator 20. As another example, if at least one electromagnetic field generator 20 is attached to the face of patient 21, the tracker module 250 generates the position and orientation information representing movement of the face from the digitized signals derived from electromagnetic signals that are generated by the at least one electromagnetic field generator 20. The position and orientation information representing a portion, such as the face, of patient 21 is different than the position and orientation information representing another portion, such as the limbs, of patient 21.

In one embodiment, system controller 210 determines a moving portion of patient 21 from the position and orientation information representing a position and an orientation of the portion and from a unique identifier of the at least one electromagnetic field generator 20 attached to the portion. For example, system controller 210 determines that there is a change in position and orientation of a leg of patient 21 upon receiving a signal representing a change in the position and orientation of at least one electromagnetic field generator 20 attached to the leg of patient 21 and determining that the attachment of the at least one electromagnetic field generator 20 to the leg is represented by a unique identifier.

In another embodiment, system controller 210 determines a particular moving portion of patient 21 from a magnitude of a digitized signal derived from the electromagnetic signal sensed by the at least one electromagnetic sensor 22. For example, upon determining that a magnitude of a digitized signal derived from the electromagnetic signal sensed by the at least one electromagnetic sensor 22 exceeds a limit, system controller 210 determines that the electromagnetic signal is generated from the at least one electromagnetic sensor 22 attached to a leg of patient 21. As another example, upon determining that a magnitude of a digitized signal derived from the electromagnetic signal sensed by the at least one electromagnetic sensor 22 does not exceed the limit, system controller 210 determines that the electromagnetic signal is generated from the at least one electromagnetic sensor 22 attached to another portion, such as a face or neck, of patient 21.

System controller 210 records the virtual stickman or atlas based representation within memory 220. In another embodiment, system controller 210 controls disk controller 240 to record the atlas based representation or the virtual stickman within disk 245. System controller 210 accesses any portion of the atlas based representation or the virtual stickman from memory 220 and/or from disk 245 upon receiving a command to access the portion recorded at a particular time within memory 220 and/or from disk 245.

In an embodiment, tracker module 250 provides the position and orientation information to a display controller 230 over local interface 215. The display controller 230 is used to output the position and orientation information on at least one display 214. The display controller 230 may output the virtual stickman or the atlas based representation on at least one display 214. The virtual stickman and the atlas based representation are not images of patient 21 acquired by using a video camera.

In another embodiment, the position and orientation information is downloaded from memory 220 via a medium, such as a wired medium, a wireless medium, a universal serial bus (USB) interface, the Internet, Intranet, or SneakerNet, by a remote computer located at a remote location relative to a location of memory 220. The remote computer may be located at a clinic, a physician's home, a physician's office, or a hospital when memory 220 may be located at a home of patient 21.

From the position and orientation information received from tracker module 250, system controller 210 creates an event profile of patient 21 to identify an activity or event, such as coughing, respiratory cycle, sneezing, scratching, restless leg syndrome, or any movement during sleep, occurring in patient 21. For example, system controller 210 generates the atlas based representation of body poses of patient 22. If the atlas based representation matches or is within a threshold of a pre-stored representation within memory 220 or disk 245, the system controller 210 determines that patient 21 has the restless leg syndrome. As another example, if the atlas based representation matches or is within a range of a pre-set representation within memory 220 or disk 245, the system controller 210 determines that patient 21 is coughing. As yet another example, if system controller 210 determines that patient 21 is experiencing a certain pre-stored frequency, stored within memory 220 or disk 245, of events of the restless leg syndrome, system controller 210 determines that patient 21 has sleep apnea. The user provides the pre-stored representation, the pre-set representation, and the pre-stored frequency to memory 220 or disk 245 via an input device (not shown), such as a mouse or a keyboard, connected to local interface 215.

In another embodiment, the user determines the activity of patient 21 from the atlas based representation of the virtual stickman. For example, upon determining that at least one display 214 displays a supine or prone position of patient 21 represented in the atlas based representation and upon determining a change in position and orientation of the at least one electromagnetic field generator 20 attached to a leg of patient 21, the user determines that patient 21 has the restless leg syndrome. As another example, if the user determines at least one display 214 displays sudden movements of patient 21 with hands towards the mouth of patient 21 in the atlas based representation, the user determines that patient 21 is coughing. As yet another example, if the user determines at least one display 214 frequently displays sudden movements of patient 21 with hands towards the mouth of patient 21 in the atlas based representation, the user determines that patient 21 has sleep apnea.

In yet another embodiment, system controller 210 receives the position and orientation information to generate a statistical analysis, such as a variation or a standard deviation, of the position and orientation information or of a plurality of events changing over time. System controller 210 may filter out a portion of the position and orientation information, such as a location of at least one electromagnetic field generator 20 with respect to at least one electromagnetic field sensor 22, that is outside a range, pre-stored within disk 245 or memory 220, of location of at least one electromagnetic generator 20 with respect to at least one electromagnetic field sensor 22. In yet another embodiment, system controller 210 generates a graph representing the position and orientation information or the statistical analysis, and a display controller 230 displays the graph on at least one display 214. The user provides the pre-stored range to memory 220 or disk 245 via the input device (not shown).

In still another embodiment, system controller 210 generates the event profile or a correlation between a time of occurrence of an event and the event. Display controller 230 displays the time-event correlation on at least one display 214. The user offers a preliminary diagnosis to patient 21 based on the event profile. A plurality of parameters, such as placement within room, range of electromagnetic field generated by at least one electromagnetic field generator 20, range of electromagnetic field sensed by at least one electromagnetic field sensor 22, are determined based on the event and/or the statistical analysis. For example, if system controller 210 determines that patient 21 has the restless leg syndrome, system controller 210 determines to place a pre-stored number of at least one electromagnetic sensor 22 on table 30 supporting the patient 21. The user provides the pre-stored number of at least one electromagnetic sensor 22 to memory 220 or disk 245 via the input device (not shown).

The position and orientation information is used not only to diagnose and monitor a sleep disorder, such as sleep apnea, but also time and control an appropriate therapy. System controller 210 times and controls an appropriate therapy delivered by a therapy system 290, such as a sleep disorder sensing device or an interventional device, based upon the event or the event profile. As an example, upon determining that patient 21 is experiencing a sleep disorder or irregular breathing, system controller 210 sends a signal to a driver that opens a valve of therapy system 290 to provide a supply of oxygen to patient 21. As another example, upon determining that patient 21 has the restless leg syndrome, system controller 210 sends a signal to an electrode of therapy system 290, attached to patient 21, to provide an electric shock to patient 21. Examples of the therapy system 290 includes a continuous positive airway pressure (CPAP) system, a drug delivery system, an electrocardiogram machine, a blood oxygen sensing system, an alarm system, an electrode stimulation system having a plurality of electrodes, as well as combinations thereof. In another embodiment, the therapy system 290 is integrated with medical navigation system 110.

In yet another embodiment, the system controller 210 determines in conjunction with the therapy system 290 whether to trigger or activate a therapy provided by the therapy system 290. For example, upon determining from the position and orientation information that patient 21 is experiencing a sleep disorder and upon determining that therapy system 290 has determined the sleep disorder, system controller 210 controls the therapy system 290 to provide a drug, oxygen, or an electrical shock, to patient 21.

It should be noted that according to alternative embodiments, at least one electromagnetic sensor 22 may be an electromagnetic receiver, an electromagnetic transceiver, or an electromagnetic generator. Likewise, it should be appreciated that according to alternate embodiments, at least one electromagnetic field generator 20 may be an electromagnetic receiver, an electromagnetic transceiver, or an electromagnetic transmitter.

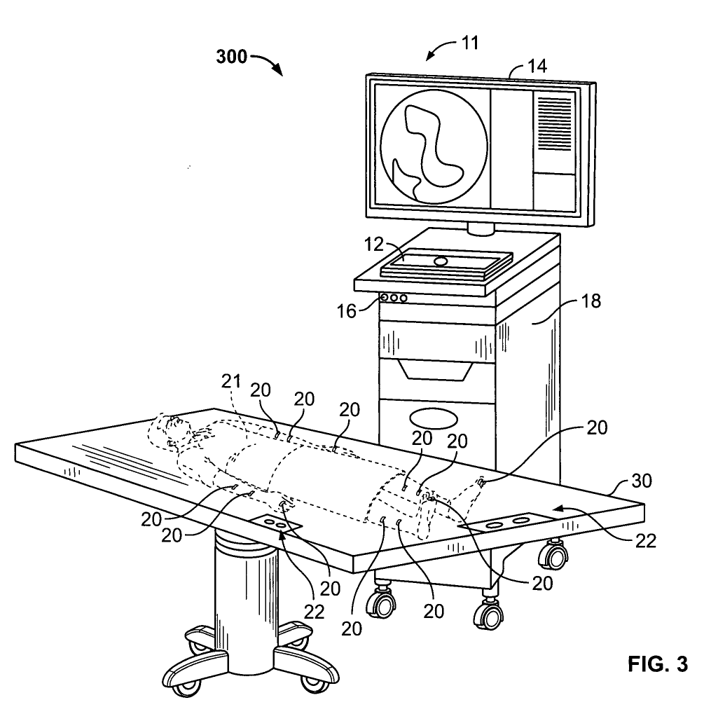

FIG. 3 is an isometric view of an embodiment of a system 300 for monitoring an activity of patient 21. System 300 includes medical navigation system 11. System 300 further includes at least one electromagnetic field generator 20 and at least one electromagnetic field sensor 22. At least one electromagnetic field generator 20 is attached to limbs of patient 21. In other embodiments, at least one electromagnetic field generator 20 is attached to a chin, torso, head, a finger, thighs, neck, chest, forehead, or abdomen, of patient 21.

Moreover, at least one electromagnetic field sensor 22 is attached to a side portion and a bottom portion of table 30 compared to that shown in the embodiment of FIG. 1 where at least one electromagnetic field sensor 22 is located on a top portion of table 30. At least one electromagnetic field sensor 22 receives electromagnetic field signals generated by at least one electromagnetic field generator 20.

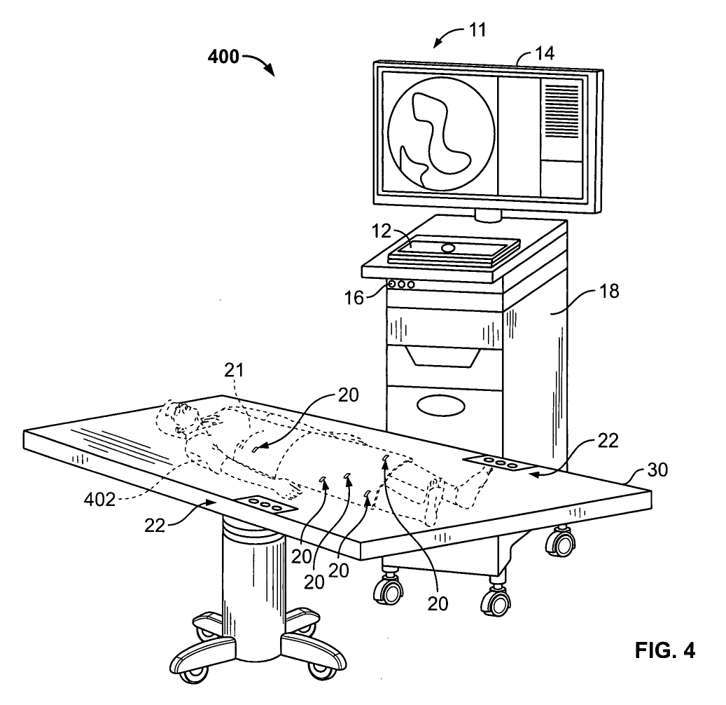

FIG. 4 is an isometric view of an embodiment of a system 400 for monitoring an activity of patient 21. System 400 includes medical navigation system 11. Moreover, system 400 includes at least one electromagnetic field generator 20 that is embedded or implanted within a garment 402, such as a long shirt, worn by patient 21. Other examples of a garment worn by patient 21 include a shirt, a t-shirt, a pyjamas, or a trouser. In another embodiment, at least one electromagnetic field generator 20 is attached, via an adhesive or Velcro™, to garment 402. In yet another embodiment, at least one electromagnetic field generator 20 is attached to a plurality of pins or a plurality of cuffs worn by patient 21. At least one electromagnetic field generator 20 generates an electromagnetic field that is received or sensed by at least one electromagnetic field sensor 22.

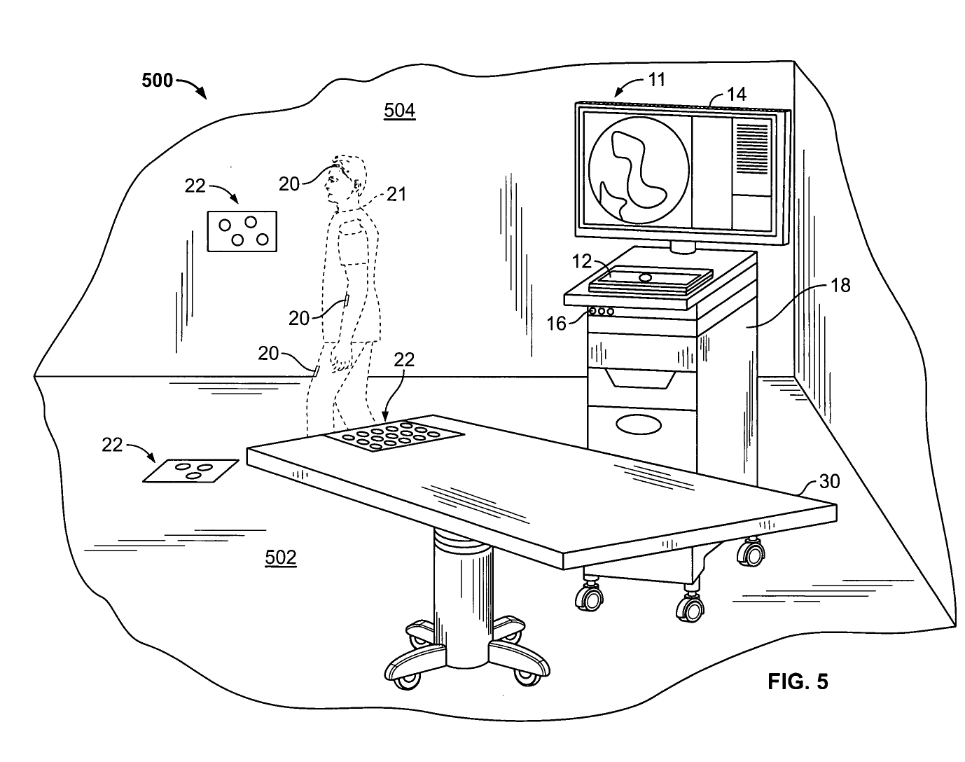

FIG. 5 is an isometric view of an embodiment of a system 500 for monitoring an activity of patient 21. System 500 includes medical navigation system 11. Moreover, system 500 includes at least one electromagnetic field generator 20 and at least one electromagnetic field sensor 22. At least one electromagnetic field sensor 22 is attached to a floor 502 of a room, such as a hospital room, an emergency room, a room of a house of patient 21, or a room in a clinical facility. Moreover, at least one electromagnetic field sensor 22 is attached to a wall 504 of the room. In another embodiment, at least one electromagnetic field sensor 22 is attached to a bedside, such as to medical navigation system 11, to a secondary monitoring device, such as an electrocardiogram machine, or to therapy system 290. At least one electromagnetic field generator 20 generates an electromagnetic field that is detected by at least one electromagnetic field sensor 22. Medical navigation system 11 determines an activity of patient 22, who has the restless leg syndrome, inside the room from digitized signals representing the electromagnetic field.

Technical effects of the herein described systems and methods for monitoring an activity of patient 21 include determining whether patient 21 is experiencing an even of sleep disorder based on the electromagnetic signal sensed by at least one electromagnetic field sensor 22. Other technical effects include activating therapy system 290 to provide a therapy to patient 21 upon determining that patient 21 is experiencing an event of sleep disorder. Yet other technical effects include tracking a movement of patient 21 from supine to prone position and vice versa based on the atlas based representation. Still other technical effects include removing a need for a presence of the user in the same room as patient 21 to monitor the event occurring in patient 21. Other technical effects include overcoming line-of-sight restrictions in an optical tracking system. A line-of-sight is not needed between at least one electromagnetic field generator 20 and at least one electromagnetic field sensor 22. For example, at least one electromagnetic field sensor 22 receives signals from at least one electromagnetic field generator 20 when patient 21 is lying on a prone position on table 30 and the at least one electromagnetic field generator 20 is located between patient 21 and table 30. As another example, at least one electromagnetic field sensor 22 receives electromagnetic signals from at least one electromagnetic field generator 20 when patient 21 is wearing the garment and the at least one electromagnetic field generator 20 is located inside the garment. The electromagnetic signals pass through the garment.

Moreover, a plurality of oncology applications can be used in synergy with the systems and methods for monitoring an activity of patient 21. Yet other technical effects include providing a level of comfort and privacy to patient 21 by generating the atlas based representation or the virtual stickman based on the electromagnetic signals sensed by the at least one electromagnetic field sensor 22. Still other technical effects include providing a representation of position and orientation of any portion of patient 21 at any desired time by recording the atlas based representation or the virtual stickman of patient 21 and instantaneously accessing any portion of the atlas based representation or the virtual stickman. Other technical effects include determining a movement of a particular portion, such as hands, legs, or face, of patient 21.

While the invention has been described in terms of various specific embodiments, those skilled in the art will recognize that the invention can be practiced with modification within the spirit and scope of the claims.

What is claimed is:

1. A system for monitoring an activity of a patient, said system comprising:
at least one electromagnetic transmitter configured to transmit at least one electromagnetic signal;
at least one electromagnetic receiver configured to receive the at least one electromagnetic signal; and
a processor configured to determine at least one position and at least one orientation of said at least one electromagnetic transmitter with respect to said at least one electromagnetic receiver to determine the activity of the patient.

2. A system in accordance with claim 1, wherein the activity includes a sleep disorder.

3. A system in accordance with claim 1 further comprising a display device configured to display a movement of the patient based on the at least one electromagnetic signal.

4. A system in accordance with claim 1, wherein the activity represents one of an event of coughing, an event of sneezing, an event of scratching, and an event of restless leg syndrome.

5. A system in accordance with claim 1 further comprising a display device configured to display, based on the at least one electromagnetic signal, at least one position and at least one orientation of said at least one electromagnetic transmitter with respect to said at least one electromagnetic receiver.

6. A system in accordance with claim 1 further comprising a display device configured to display, based on the at least one electromagnetic signal, at least one position and at least one orientation of said at least one electromagnetic transmitter with respect to said at least one electromagnetic receiver to an operator, wherein the operator manually determines whether the patient is experiencing an event of sleep apnea based on the at least one position and at least one orientation.

7. A system in accordance with claim 1, wherein said processor is configured to generate an atlas based representation of a plurality of body poses of the patient based on the at least one electromagnetic signal.

8. A system in accordance with claim 1 further comprising a therapy system, wherein said processor triggers said therapy system to deliver a therapy to the patient.

9. A system in accordance with claim 1, wherein said processor configured to generate a statistical analysis from the at least one position and at least one orientation.

10. A system for monitoring an activity of a patient, said system comprising:
at least one electromagnetic transmitter configured to transmit at least one electromagnetic signal;
at least one electromagnetic receiver configured to receive the at least one electromagnetic signal; and
a processor configured to determine at least one mutual inductance between said at least one electromagnetic transmitter and said at least one electromagnetic receiver, wherein said processor configured to determine at least one position and at least one orientation of said at least one electromagnetic transmitter with respect to said at least one electromagnetic receiver based on the at least one mutual inductance to determine the activity of the patient.

11. A system in accordance with claim 10, wherein the activity represents a sleep disorder.

12. A system in accordance with claim 10 further comprising a display device configured to display a movement of the patient based on the at least one electromagnetic signal.

13. A system in accordance with claim 10, wherein the activity represents one of an event of coughing, an event of sneezing, an event of scratching, and an event of restless leg syndrome.

14. A system in accordance with claim 10 further comprising a display device configured to display, based on the at least one electromagnetic signal, at least one position and at least one orientation of said at least one electromagnetic transmitter with respect to said at least one electromagnetic receiver.

15. A system in accordance with claim 10 further comprising a display device configured to display, based on the at least one electromagnetic signal, at least one position and at least one orientation of said at least one electromagnetic transmitter with respect to said at least one electromagnetic receiver to an operator, wherein the operator manually determines whether the patient is experiencing an event of sleep apnea based on the at least one position and orientation.

16. A method for monitoring an activity of a patient, said method comprising:
transmitting at least one electromagnetic signal from at least one electromagnetic transmitter;
receiving, by at least one electromagnetic receiver, the at least one electromagnetic signal; and
determining the activity of the patient by determining at least one position and at least one orientation of the at least one electromagnetic transmitter with respect to the at least one electromagnetic receiver.

17. A method in accordance with claim 16, wherein the activity represents a sleep disorder.

18. A method in accordance with claim 16 further comprising displaying a movement of the patient based on the at least one electromagnetic signal.

19. A method in accordance with claim 16, wherein the activity represents one of an event of coughing, an event of sneezing, an event of scratching, and an event of restless leg syndrome.

20. A method in accordance with claim 16 further comprising displaying, based on the at least one electromagnetic signal, at least one position and at least one orientation of the at least one electromagnetic transmitter with respect to the at least one electromagnetic receiver.
